(19) United States
(12) Patent Application Publication
(10) Pub. No.: US 2022/0229037 A1
(43) Pub. Date: Jul. 21, 2022

(54) APPARATUS AND METHOD FOR DETERMINING FILMING AMINE CONCENTRATION IN WATER

(71) Applicant: Waltron Bull & Roberts, LLC, Toronto, NJ (US)

(72) Inventors: Harold W. STANSFIELD, Southampton, PA (US); Eric ETTER, Doylestown, PA (US)

(73) Assignee: Waltron Bull & Roberts, LLC, Toronto, NJ (US)

(21) Appl. No.: 17/615,399

(22) PCT Filed: Apr. 23, 2020
(86) PCT No.: PCT/US2020/029628

§ 371 Date: Nov. 30, 2021

Related U.S. Application Data
(60) Provisional application No. 62/837,588, filed on Apr. 23, 2019.

(51) Int. Cl. G01N 33/18; G01N 21/78

(52) U.S. Cl. CPC G01N 33/1826; G01N 2021/7783; G01N 21/78

(57) ABSTRACT

The present application provides an apparatus and method of determining water filming amine concentration, which includes performing a measuring cycle and a cleaning cycle. The measuring cycle includes providing sample water to a sample water measuring container, providing, to the sample water measuring container, one or more reaction chemicals which generate color in the sample water, emitting light, via a light emitter, at a wavelength range and intensity range, through the sample water having the generated color, to a light receiver, receiving an indication of a light intensity of the light emitted through the sample water and determining a filming amine concentration of the sample water based on the light intensity. The cleaning cycle includes providing a cleaning reagent to remove amines from the sample water measuring container.

### CROSS REFERENCE TO RELATED APPLICATION

This application claims the benefit of U.S. Provisional Application No. 62/837,588, filed on Apr. 23, 2019, the contents of which are hereby incorporated by reference herein.

### SUMMARY

The present application discloses apparatuses and methods for detecting the concentration of filming amines film-forming amines (FFA) (hereinafter filming amines) in water.

The present application provides a method of determining water filming amine concentration, which includes performing a measuring cycle and a cleaning cycle. The measuring cycle includes providing sample water to a sample water measuring container, providing, to the sample water measuring container, one or more reaction chemicals which generate color in the sample water, emitting light, via a light emitter, at a wavelength range and intensity range, through the sample water having the generated color, to a light receiver, receiving an indication of a light intensity of the light emitted through the sample water and determining a filming amine concentration of the sample water based on the light intensity. The cleaning cycle includes providing a cleaning reagent to remove amines from the sample water measuring container.

The present application provides an apparatus for determination water filming amine concentration is provided which includes a measuring cell portion. The measuring cell portion includes a sample water container configured to receive and hold sample water, a light emitter configured to emit light, at a wavelength range and an intensity range, through the sample water and a light receiver configured to receive an indication of a light intensity of the light emitted through the sample water. The apparatus also includes a sample provider portion which includes a sample water selection valve configured to cause a flow path of the sample water to change between different sample water sources, a pump configured to provide the sample water to the measuring cell portion. The apparatus also includes a reagent provider portion comprising a plurality of reagent providers, each configured to provide, to the measuring cell portion, one of a reaction reagent which generates color in the sample water and a cleaning reagent which removes filming amines from the sample water measuring container. The apparatus also includes a processing device having one or more processors configured to receive an indication of a light intensity of the light emitted through the sample water, determine a filming amine concentration of the sample water based on the light intensity and provide a cleaning reagent to remove amines from the sample water measuring container.

### BRIEF DESCRIPTION OF THE DRAWINGS

A more detailed understanding can be had from the from the following description, given by way of example in conjunction with the accompanying drawings wherein:

FIG. 1 is a block diagram of an example system used to determine filming amine water concentration according to embodiments disclosed herein; and

FIG. 2 is a flow diagram illustrating an example method of determining filming amine water concentration according to embodiments described herein.

### DETAILED DESCRIPTION

Filming amines are a common product used in steam powered power plants as a water treatment product. These products are long-chain organic amines, such as for example octodecyl-amine (CH₃(CH₂)₁₆CH₂NH₂). Filming amines provide excellent corrosion control, particularly in cycling power plants where there is exposed metal during down time.

Conventional systems and methods of monitoring the presence of amines in water cycles during operation include continuously injecting the amines into the water cycle and monitoring the amines through labor intensive, manual measurements. Reliable measurements of the filming amines require the use of expensive and highly technical equipment, however, such as mass spectroscopy or high pressure liquid chromatography. More simplified methods of monitoring amines in water cycles are highly technical (requiring hours of training), however, and are not considered very reliable.

Automated systems (i.e., systems in direct communication with the water cycle of the power plant) for determining amine concentration in water can reduce or eliminate manual labor and costly equipment requirements of conventional systems and methods. Automated filming amine analyzers, however, encounter additional challenges, due in part to the prevalence of multiple proprietary film forming amine formulas with various reaction intensities and, due to their nature, the coating that filming amines leave on the measuring and analysis equipment.

The present application discloses automated systems and methods disclosed which efficiently determine accurate filming amine concentration measurements in water while reducing or eliminating the manual labor and costly equipment requirements of conventional systems and methods. The amine concentration is determined by generating, via reactant reagents, color to the sample water and measuring, via optical measurement devices (e.g., light emitter, light receiver), a light intensity range of light in a specific wavelength range passing through the sample water. A non-ionic surfactant cleaning reagent is provided to remove filming amines from a sample water measuring container to prevent interference with optical measurements to determining accurate amine concentration, which would otherwise occur from amines remaining on the container. Systems and methods disclosed herein utilize the Lambert-Beer relationship between the absorbance of a material to the path length through it and the concentration of the material being examined.

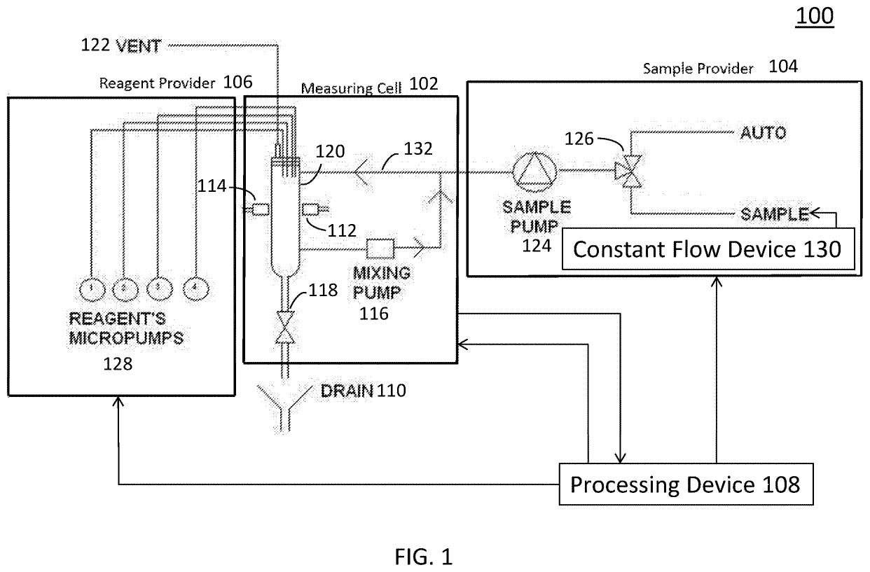

FIG. 1 is a block diagram of an example system used to determine filming amine water concentration according to embodiments disclosed herein.

As shown in FIG. 1, the example system 100 includes a measuring cell portion 102, a sample provider portion 104, a reagent provider portion 106, a processing device 108 and a drain 110.

As shown in the example at FIG. 1, the measuring cell portion 102 includes a light emitter 112, a light receiver 114, a mixing pump 116, valve 118, sample water container 120 (e.g., glass tube) and container vent 122. The measuring cell portion 102 may optionally include a heater (not shown). The light emitter 112 is configured to provide light, at a predetermined wavelength range and a predetermined intensity range through the sample water in the sample water container 120 to the light receiver 114. Alternatively, the light emitter is configured to emit a white light source. Examples of light emitters 114 providing a target wavelength range include light emitting diodes (LED) and an incandescent light source (e.g., an incandescent lamp).

The light receiver 114 is configured to receive the light emitted from the light emitter 112 at the predetermined wavelength and intensity ranges. The light receiver 114 is configured to acquire an optical measurement reading (e.g., light intensity value) that is proportionate to the concentration of the filming amine in the water sample. As shown in the example at FIG. 1, the light receiver 114 is located adjacent to the sample water container 120 and opposite the light emitter 112. The size, shape and location of the light emitter 112 and light receiver 114 in FIG. 1 are exemplary. Examples of light receivers 114 include LEDs and vacuum phototubes. Additional measuring devices, such as light filters, may also be used to measure the light intensity range of the light passing through the sample water. For example, filters may be used to filter white light from a white light source or light from incandescent light source.

The sample water container 120, or a portion of the sample water container 120 is transparent (e.g., glass) such that the light emitted from light emitter 112 passes through the sample water in the sample water container 120 and is received by the light receiver 114.

The mixing pump 116 is configured to circulate the sample water via water mediums 132 (e.g., pipes) such that the coloring reagents are mixed into the water during the measurement cycle and such that the cleaning reagents are mixed into the water during a cleaning cycle. Although a single mixing pump 116 is shown in FIG. 1, embodiments include separate mixing pumps and pipes for mixing during the measurement and cleaning cycles.

As shown in the example at FIG. 1, the sample provider portion 104 includes a water pump 124, a 3-way valve 126 (i.e., sample selection valve) and a constant flow device 130.

The constant flow device 130 is configured to provide water (i.e., sample water), from the water cycle of the power plant, to the sample provider portion 104 and the measuring cell 102 at a constant flow range. In some embodiments, the constant flow device 130 is housed within or integrated with a single apparatus (i.e., sample provider portion 104) along with the sample pump 124 and the 3-way valve 126. In other embodiments, the constant flow device 130 is coupled to, but separate from the components of the sample provider portion 104.

The 3-way valve 126 is controlled (e.g., by a processor of processing device 108) such that the sample water is provided from the water cycle via the constant flow device 130 (along the path indicated by SAMPLE in FIG. 1) to be the measuring cell portion 102. Alternatively, the 3-way valve 126 is controlled such that water is provided from a container (not shown) having a known amine concentration (e.g., predetermined amine concentration provided to a processor of processing device 108) such that the processing device 108 can determine that the components of the system 100 are providing accurate amine concentration measurements (e.g., to processing device 108). The 3-way valve 126 may be controlled to cause the water to flow along the SAMPLE path or AUTO path at predetermined time intervals, upon request (e.g., by an operator) or upon the occurrence of an event.

As shown in the example at FIG. 1, the reagent provider portion 106 includes reagent micropumps 128 and reagent mediums 134 (e.g., pipes, tubes and the like). In the example shown in FIG. 1, four micropumps (1), 128(2), 128(3) and 128(4) are used. The number of micropumps shown in FIG. 1 is exemplary. Any number of micropumps 128 may be used to provide cleaning reagents and coloring reagents to the sample water container 120. For example, each micropump 128 is configured to provide the cleaning reagent or one of a plurality of reacting reagents (e.g., coloring) reagents, from one of a plurality of reagent containers (not shown), to the sample water container 120. Examples of other types of reagent providing devices (i.e., other than pumps) used to provide the reacting reagents and cleaning reagents include air compressors and vacuums. In some examples, the reagents may be provided via the mediums 134 and gravity.

The chemical reagents are configured to allow dosing into the measuring cell portion 102 up to a predetermined amount to react with the filming amines in the water sample. The mixture of these reagents with the water sample causes a reaction that causes color to form at a predetermined rate. This color formation is measured by the indication of light absorbance as it transmits through the sample in the measuring cell portion 102.

The cleaning reagent is a non-ionic surfactant which is configured to remove filming amines from a sample water measuring container to prevent interference with optical measurements to determine accurate amine concentration which would otherwise occur from amines on the container. The non-ionic surfactant is a detergent which does not include chemical amines.

The processing device 108 includes one or more processors and other components (not shown) such as memory, circuitry, wires, buses, transmitters, receivers and network interfaces. The processing device 108 may be configured to communicate (wired or wirelessly) with components of system 100. As shown in FIG. 1, processing device 108 is in communication with components of the measuring cell portion 102, the sample provider portion 104 and the reagent provider portion 106, such as light emitter 112, light receiver 114, mixing pump 116, valve 118, sample pump 124, 3-way valve 126, micropumps 128 and constant flow device 130. Additionally, one or more electronic and processing components, such as one or more additional control processors (not shown), separate from processing device 108, can be located at one or more of the components of the system 100 and configured to communicate with processing device 108. Processing device 108 may receive data from components of system 100 and control components of system 100 directly from the components or indirectly via the one or more additional control processors.

For example, processing device 108 performs one or more functions, such as: receiving data via light receiver 114 and determining, from the data, the amine concentration of the sample water from the water cycle of the plant when the sample water is received along the SAMPLE path and the known amine concentration when the sample water is received along the AUTO path; controlling light emitter 112 to emit the light to be received by the light receiver 114, controlling mixing pump 116 to pump the water via mediums 132; controlling valve 118 to close and hold the water and reagents in the sample water container 120 during operation of the measuring and cleaning cycles and open and drain the water after each cycle; controlling 3-way valve 126 to open and close the SAMPLE and AUTO paths such that the sample water is received along the SAMPLE path or the sample water is received along the AUTO path; controlling constant flow device 130 to provide the water, at a constant flow range, to the sample provider 104 and measuring cell 102; controlling sample pump 124 to pump the water, via the SAMPLE path and the AUTO path, to the measuring cell 102; and controlling micropumps 128 to provide the cleaning reagents and coloring reagents to the measuring cell 102.

The processing device 108 is also configured to receive user input via user input devices, such as a touch screen (not shown). Processing device 108 is also configured to process instructions (e.g., from user input and predefined programmed instructions) and the data received from the measuring cell portion 102 to perform one or more of the functions described herein to determine the amine concentration in the water. Processing device 108 may also be configured to provide (e.g., display) amine concentration measurements to a user via a display device (not shown).

The processing device 108 is configured to receive an indication of the absorbance of the light source passing through by the sample water and to calculate the concentration of filming amines in the sample water using the Lambert-Beer Law.

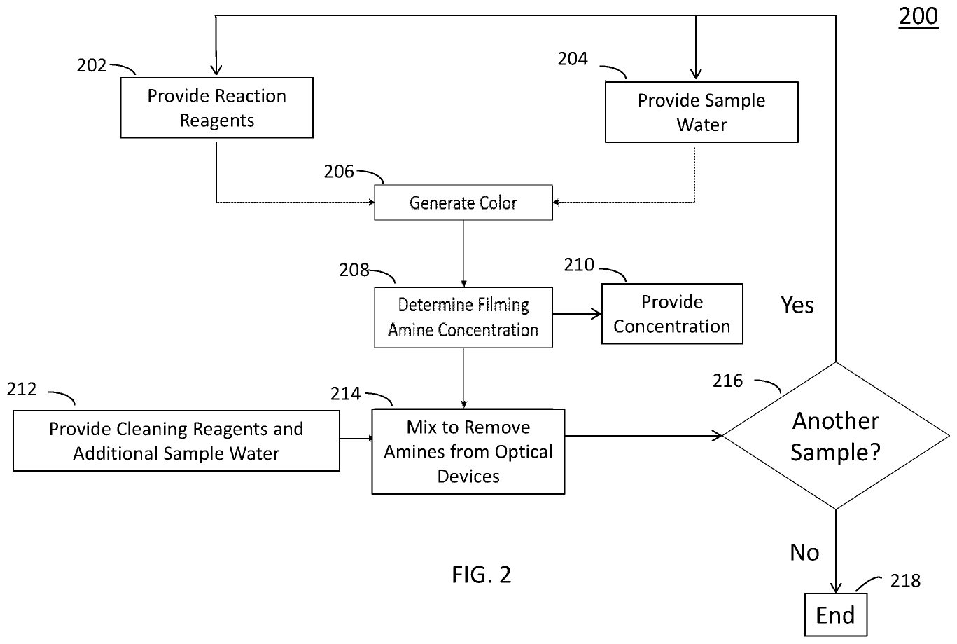

FIG. 2 is a flow diagram illustrating an example method of determining filming amine concentration in water according to embodiments described herein.

As shown at block 202 in FIG. 2, the method 200 includes providing (e.g., to the sample water container 120), one or more reaction reagents (e.g., cleaning reagents or coloring reagents). A predetermined amount (e.g., dosage) of one or more reaction reagents is provided to the sample water container 120 to generate color in the sample water.

As shown at block 204, in FIG. 2, the method 200 includes providing (e.g., to the sample water container 120), the sample water (e.g., the sample water from the water cycle of the plant via the SAMPLE path or the sample water, having the known amine concentration, along the AUTO path).

In one example, prior to the reagent chemicals being provided to the sample water container 120, the sample water is provided to the sample water container 120 and a first reading (e.g., light intensity value) is acquired (e.g., by light receiver 114). The one or more reaction reagents are then provided to the sample water container 120 to generate color in the sample water, as shown at block 206. After a period of time has elapsed (e.g., after the one or more reaction reagents are provided), a second reading is acquired (e.g., by light receiver 114).

In another example, the reagent chemicals and the sample water are concurrently provided to the sample water container 120 and a reading (e.g., light intensity value) is acquired (e.g., by light receiver 114). That is, the light emitter 112 provides the light in a wavelength and intensity range through the reacted sample water and the intensity of the light received is acquired by the light receiver 114. In yet another example, the reagent chemicals are provided to the sample water container 120 prior to the sample water.

A predetermined amount of one or more conditioning reagents may also be provided to the sample water to condition the sample water (e.g., condition to a predetermined pH range, (e.g., a range of 3.0 to 5.0)) to accurately measure the filming amine concentration in the sample water.

As shown at block 208, the filming amine concentration of the sample water is determined. For example, the reading (in the first example described above) or the first and second readings (in the second example described above) are sent to the processing device 108, and the processing device 108 determines the concentration of filming amines in the water sample based on the data (i.e., the reading or the first and second readings) acquired from the light receiver. The determined filming amine concentration is then provided, as shown at block 210. In other examples, additional readings may be includes, such as a reading after a condition reagent is provided, but before the reaction reagent is provided.

In some embodiments, the determined (i.e., measured) filming amine concentration is compared to another value. For example, when the sample water is received along the SAMPLE path, the light intensity from the light receiver 114 is compared to an intensity threshold (e.g., detection limit value). The result of the comparison is provided (e.g., displayed to an operator), as shown at block 210. If the light intensity from the light receiver 114 is equal to or greater than the intensity threshold, then it is determined (e.g., by processing device 108) that the filming amine concentration is detected, and the result is provided at block 210. If the light intensity from the light receiver 114 is less than the intensity threshold, however, then it is determined that the filming amine concentration is below the detection limit value, even if some amine concentration is present in the sample water, and an indication is provided, at block 210, that the filming amine concentration is below the detection limit value or not detected (e.g., a value of “0” is returned).

When the sample water is received along the AUTO path (i.e., the amine concentration when the sample water is known), the light intensity from the light receiver 114 is compared (e.g., by processing device 108) to a light intensity proportionate to the predetermined filming amine concentration. The result of the comparison is provided (e.g., displayed to an operator), as shown at block 210. For example, if the light intensity from the light receiver 114 is determined to equal to or within a threshold range of the light intensity proportionate to the predetermined filming amine concentration, then it is determined that measured concentration is accurate, which is provided at block 210. If the light intensity from the light receiver 114 is determined to not be equal to or within a threshold range of the light intensity proportionate to the predetermined filming amine concentration, then it is determined that measured concentration is not accurate, which is provided at block 210.

In one example, a precision value of the measurements over time is also determined. The value is a relative precision value within (i.e., +/−) a number (e.g., 2) of standard deviations of a bell curve determined by comparing reference values (e.g., threshold values or predetermined values) with measured values over time. For example, a relative precision value may be the larger of +/−5 ppb (parts per billion) or 1% of the measured readings.

Blocks 202-208 represent a measuring cycle of the method 200. Blocks 210 and 212 represent a measuring cycle of the method 200.

After the concentration of filming amines in the water sample is determined at the completion of the measuring cycle, the reacted and measured sample water is drained and the sample water container 120 and water mediums 132 in the measuring cell portion 102 are rinsed and the method 200 proceeds to the cleaning cycle in blocks 210 and 212.

As shown at block 212, one or more cleaning reagents (e.g., a non-ionic surfactants) and additional sample water is provided to the measuring cell portion 102, where they are mixed together remove filming amines from a sample water measuring container, as shown at block 214. This prevents interference with optical measurements to determining accurate amine concentration, which would otherwise occur from amines remaining on the container.

After a predetermined amount of time has passed, the additional sample water mixed with the cleaning reagent is also drained and the sample water container 120 and water mediums 132 in the measuring cell portion 102 are rinsed. When the cleaning cycle is completed, it is determined (e.g., by processing device 108), at decision block 216, whether another sample is being provided to determine the filming amine concentration in the water. If another sample is not being provided, the method ends at block 218. If another sample is being provided, the method proceeds back to blocks 202 and 204.

In the example shown at FIG. 2, the cleaning cycle is executed after each measuring cycle. In other examples, however, the cleaning cycle may not be executed after each measuring cycle. For example, the cleaning cycle may be executed after a predetermined number of measuring cycles has executed, after a predetermined amount of time has elapsed since the last cleaning cycle, upon request (e.g., by an operator) or upon the occurrence of an event (e.g., when it is determined that accurate measurements are not provided after a measuring cycle using the sample water along the AUTO path, or when an indication is received that a measuring device is not working correctly.

The methods provided can be implemented in a general purpose computer, a processor, or a processor core. Suitable processors include, by way of example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine. Such processors can be manufactured by configuring a manufacturing process using the results of processed hardware description language (HDL) instructions and other intermediary data including netlists (such instructions capable of being stored on a computer readable media). The results of such processing can be maskworks that are then used in a semiconductor manufacturing process to manufacture a processor which implements features of the disclosure.

The methods or flow charts provided herein can be implemented in a computer program, software, or firmware incorporated in a non-transitory computer-readable storage medium for execution by a general purpose computer or a processor. Examples of non-transitory computer-readable storage mediums include a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

It should be understood that many variations are possible based on the disclosure herein. Although features and elements are described above in particular combinations, each feature or element can be used alone without the other features and elements or in various combinations with or without other features and elements.

1. A method of determining water filming amine concentration, the method comprising:
performing a measuring cycle comprising;
providing sample water to a sample water measuring container;
providing, to the sample water measuring container, one or more coloring reagents which generate color in the sample water;
emitting light, via a light emitter, at a wavelength range and intensity range, through the sample water having the generated color, to a light receiver;
receiving an indication of a light intensity of the light emitted through the sample water; and
determining a filming amine concentration of the sample water based on the light intensity; and
performing a cleaning cycle comprising providing a cleaning reagent to remove amines from the sample water measuring container.

2. The method of claim 1, wherein determining the filming amine concentration of the sample water is further based on an absorbance of the light emitted through the sample water.

3. The method of claim 1, wherein the measuring cycle is performed between the completion of the cleaning cycle and the start of another measuring cycle.

4. The method of claim 1, wherein the sample water comprises a predetermined filming amine concentration, and
the method further comprises:
comparing the light intensity to a light intensity proportionate to the predetermined filming amine concentration, and
determining whether the filming amine concentration is within an accuracy threshold range.

5. The method of claim 1, wherein the sample water is provided directly from a power plant water cycle, and
the method further comprises:
comparing the light intensity from the light receiver to an intensity threshold, and
determining whether the filming amine concentration is detected based on the comparison.

6. The method of claim 1, further comprising providing a predetermined amount of one or more conditioning reagents to the sample water to condition the sample water to have a pH within a predetermined pH range between 3.0 and 5.0.

7. The method in claim 1, further comprising rinsing the sample water container and one or more water mediums used to provide pathways for the sample water.

8. An apparatus for determination water filming amine concentration, the apparatus comprising:
a measuring cell portion comprising:
a sample water container configured to receive and hold sample water;
a light emitter configured to emit light, at a wavelength range and an intensity range, through the sample water;
a light receiver configured to receive an indication of a light intensity of the light emitted through the sample water; and
a sample provider portion comprising:
a sample water selection valve configured to cause a flow path of the sample water to change between different sample water sources;
a pump configured to provide the sample water to the measuring cell portion;
a reagent provider portion comprising a plurality of reagent providers, each configured to provide, to the measuring cell portion, one of a coloring reagent which generates color in the sample water and a cleaning reagent which removes filming amines from the sample container; and
a processing device having one or more processors configured to:
receive an indication of a light intensity of the light emitted through the sample water;
determine a filming amine concentration of the sample water based on the light intensity; and
provide a cleaning reagent to remove amines from the sample water measuring container.
